(19) United States
(12) Patent Application Publication
(10) Pub. No.: US 2026/0124453 A1
(43) Pub. Date: May 7, 2026

(54) ACOUSTIC ANALYSIS FOR PHRENIC NERVE STIMULATION

(71) Applicant: COVIDIEN LP, Mansfield, MA (US)

(72) Inventors: Abhijit Bhattacharya, Hyderabad (IN); Lilian Kornet, Eijsden (NL); Xusheng Zhang, Shoreview, MN (US)

(73) Assignee: COVIDIEN LP, Mansfield, MA (US)

(21) Appl. No.: 19/117,890

(22) PCT Filed: Sep. 29, 2023
(86) PCT No.: PCT/US2023/075530

§ 371 Date: Apr. 2, 2025

Related U.S. Application Data
(60) Provisional application No. 63/413,487, filed on Oct. 5, 2022.

(51) Int. Cl. A61N 1/36; A61B 5/00; A61B 5/08; A61B 5/11; A61B 5/113; A61B 7/00; A61B 7/04; A61M 16/00; A61N 1/39

(52) U.S. Cl. CPC A61N 1/36139; A61B 5/0803; A61B 5/1126; A61B 5/113; A61B 5/4836; A61B 5/686; A61B 5/7264; A61B 7/003; A61B 7/04; A61M 16/0003; A61M 16/024; A61N 1/36003; A61N 1/3601; A61N 1/3606; A61N 1/3615; A61N 1/36171; A61N 1/36175; A61N 1/39; A61B 2560/0462; A61B 2562/0204; A61M 2205/04; A61M 2205/054; A61M 2205/3375; A61M 2205/50; A61M 2230/005; A61M 2230/40

(57) ABSTRACT

Aspects of this disclosure describe methods and systems for evaluating phrenic nerve stimulation based on acoustics. A stimulation lead, for pacing the phrenic nerve(s), and an acoustic sensor, for detecting sounds or vibrations, may be placed in a patient. The stimulation lead may stimulate phrenic nerve(s) to cause movement of the diaphragm, resulting in an associated acoustic signal. The acoustic sensor may detect the acoustic signal associated with the diaphragm movement. Other acoustics may be detected by the acoustic sensor, such as heart sounds. The portion of the acoustic signal associated with diaphragm movement may be identified for analysis of the phrenic nerve stimulation. Stimulation parameters of the phrenic nerve stimulation may be modified or adjusted accordingly.

### CROSS-REFERENCE TO RELATED APPLICATIONS

This application claims the benefit of U.S. Provisional Application No. 63/413,487 filed Oct. 5, 2022, entitled “Acoustic Analysis for Phrenic Nerve Stimulation,” which is incorporated herein by reference in its entirety.

### INTRODUCTION

Medical ventilator systems have long been used to provide ventilatory and supplemental oxygen support to patients. These ventilators typically include a connection for pressurized gas (air, oxygen) that is delivered to the patient through a conduit or tubing. As each patient may require a different ventilation strategy, modern ventilators can be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

Long term external ventilation is typically provided to patients using positive pressure ventilation. Positive pressure ventilation is a form of artificial respiration in which a mechanical ventilator is used to deliver a controlled volume of gasses to the lungs of a patient. In contrast, in one form of negative-pressure ventilation, the diaphragm of a patient is caused to contract to cause the chest of the patient to expand during inspiration (thereby drawing air into the lungs), and the diaphragm is caused to relax to cause the chest to contract during exhalation (thereby forcing air out of the lungs). While lifesaving and valuable, positive pressure ventilation is non-physiological; that is, forcing air into the lungs is not the manner in which humans naturally breathe. Accordingly, the greater the positive pressure and/or the number of positive-pressure cycles, the more likely the patient will experience detrimental effects, such as an illness becoming more severe, ventilator-induced lung injury (VILI), acute respiratory distress syndrome (ARDS), ventilator-associated pneumonia (VAP), diaphragm dystrophy, and/or delay of ventilator weaning. These effects may increase an amount of time a patient is subjected to mechanical ventilation, leading to longer hospital stays and increased medical costs.

It is with respect to this general technical environment that aspects of the present technology disclosed herein have been contemplated. Furthermore, although a general environment is discussed, it should be understood that the examples described herein should not be limited to the general environment identified herein.

### SUMMARY

Among other things, aspects of the present disclosure include systems and methods for acoustic analysis of phrenic nerve stimulation. In an aspect, the technology relates to a method for providing phrenic nerve stimulation. The method includes delivering phrenic nerve stimulation, at a first time based on stimulation parameters, to a phrenic nerve to cause movement of a diaphragm; acquiring, from an internal acoustic sensor, an acoustic signal including contributions from diaphragm movement and cardiac movement; identifying a first portion of the acoustic signal associated with the diaphragm movement and a second portion of the acoustic signal associated with cardiac movement; based the first portion of the acoustic signal, adjusting the stimulation parameters; and delivering phrenic nerve stimulation, at a second time, based on the adjusted stimulation parameters.

In an example, the method further includes determining, based at least on the first portion of the acoustic signal, that the movement of the diaphragm caused by the phrenic nerve stimulation is associated with one of a hiccup or a low tidal volume. In still another example, adjusting the stimulation parameters is based on determining that the movement of the diaphragm is associated with one of the hiccup or the low tidal volume. In yet another example, the acoustic sensor is part of at least one of an implantable cardioverter-defibrillator (ICD), pacemaker, or neurostimulator. In still yet another example, the method further includes determining a ratio based on at least one acoustic attribute the first portion of the acoustic signal and at least one stimulation parameter of the stimulation parameters; calculating an atrophy threshold, based on a moving average of the ratio; and determining that the ratio is below the atrophy threshold; and generating a notification for diaphragm atrophy. In a further example, wherein the acoustic attribute is a peak amplitude of the first portion of the acoustic signal. In another example, the method further includes triggering an inspiratory phase of a breath based on one or more acoustic attributes of the first portion of the acoustic signal.

In another aspect, the technology relates to a method for providing phrenic nerve stimulation. The method includes acquiring, by an internal acoustic sensor, a baseline acoustic signal; delivering phrenic nerve stimulation to a phrenic nerve, based on stimulation parameters, to cause artificial movement of a diaphragm; subsequent to delivering the phrenic nerve stimulation, acquiring, by the internal acoustic sensor, a stimulated acoustic signal; comparing, by a phrenic nerve stimulation controller, the stimulated acoustic signal and the baseline acoustic signal; and identifying a portion of the stimulated acoustic signal attributable to the artificial movement of the diaphragm.

In an example, acquiring the baseline acoustic signal occurs during a period where no phrenic nerve stimulation is delivered. In another example, the method further includes adjusting at least one of the stimulation parameters based on the portion of the stimulated acoustic signal attributable to the artificial movement of the diaphragm. In a further example, the stimulation parameters include at least one of: a frequency; a timing; an amplitude; or a pulse width. In yet another example, the phrenic nerve stimulation controller is integrated into a medical ventilator

In another example, the technology relates to a phrenic nerve stimulation system including an acoustic sensor internal to a body; a phrenic nerve stimulation device; a processor; memory storing instructions that, when executed by the processor, cause the system to perform a set of operations. The operations include delivering a first phrenic nerve stimulation, via the stimulation device, to at least one phrenic nerve of the body according to stimulation parameters; acquiring, via the acoustic sensor, an acoustic signal of sounds from within the body; identifying a portion of the acoustic signal associated with diaphragm movement; based on the identified portion of the acoustic signal, adjusting the stimulation parameters; and delivering a second phrenic nerve stimulation, via the stimulation device, based on the adjusted stimulation parameters.

In an example, the system further includes an implantable cardioverter-defibrillator (ICD), wherein the acoustic sensor is part of the ICD. In another example, the system further includes
a ventilator, and the operations further include based on the identified portion of the acoustic signal, determining a reactivity time between the delivery of the first phrenic nerve stimulation and the diaphragm movement; and causing an inspiratory breath to be triggered at about the reactivity time after delivery of the second phrenic nerve stimulation.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 shows an anatomy of a human patient, including phrenic nerves, a heart, and a diaphragm.

FIG. 2 shows a stimulation device and an acoustic sensor placed in the body of the patient of FIG. 1.

FIGS. 3A-3D show graphical representations of acoustic signals.

FIG. 4 shows another graphical representation of acoustic signals.

FIG. 5 shows a method for phrenic nerve stimulation based on an acoustic signal.

FIG. 6 shows a method for timing phrenic nerve stimulation.

FIG. 7A shows another method for timing phrenic nerve stimulation.

FIG. 7B-7C show methods for synchronizing phrenic nerve stimulation with mechanical ventilation.

FIG. 8 shows a method for identifying diaphragm movement attributable to phrenic nerve stimulation.

FIG. 9 shows a method for determining diaphragm atrophy.

FIG. 10 shows a diagram illustrating an example of a ventilator connected to a patient, a stimulation device, and an acoustic sensor.

FIG. 11 shows a block-diagram illustrating an example of a ventilatory system.

FIGS. 12-13 show example stimulation systems including a stimulation device, an application tool, and a connector.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. Approximately one third of patients in the intensive care unit (ICU) depend on mechanical ventilation. With millions of people each year admitted to the intensive care unit, many people per year rely on mechanical ventilation. The amount of time a patient is assisted via mechanical ventilation may vary, as may be based on a lung condition of the patient. For example, approximately a third of patients with chronic obstructive pulmonary disease (COPD) or ARDS may seek assistance from a mechanical ventilator for longer than four days, with some patients requiring mechanical ventilation longer than seven days. As another example, patients with sleep apnea may require mechanical ventilation while sleeping, over an extended period of time. In addition, patients in deep anesthesia, patients with spinal cord injury, and patients with other heath conditions may require longer periods of ventilator-assisted breathing.

Weaning patients off mechanical ventilation may be difficult. A patient's diaphragm may begin to atrophy after as little as two days of ventilation. After a patient's diaphragm muscles begin to atrophy, a patient may require slow weaning to encourage the patient to breathe on their own (e.g., via the patient's diaphragm contracting unassisted by a mechanical ventilator). Increased time on a ventilator is associated with increased risk of infection, hyperventilation, hypoventilation, decreased venous return, and subsequent rehospitalization. Thus, minimizing a patient's time on a ventilator may be desirable for diaphragm health and/or to reduce the risk of infection and/or rehospitalization.

To help prevent or reduce the likelihood of diaphragm muscle atrophy and reduce the time that a patient is dependent on mechanical ventilation, one solution is to exercise the patient's diaphragm muscles during ventilation, or in lieu of ventilation. To exercise a patient's diaphragm, the phrenic nerve can be stimulated with an electrical current. The electrical current used to stimulate the phrenic nerve may be provided by nearby electrodes that conduct the electrical current, from a stimulation source, through the body of the patient. The electrodes may be positioned inside of the body of the patient close enough to the phrenic nerve to stimulate the phrenic nerve. For example, electrodes may be positioned on a device that is insertable into the body of the patient. In one example, electrodes may be positioned on a lead insertable into a blood vessel (e.g., vein, artery, arteriole, capillary, venule, etc.) near a phrenic nerve or in a structure of a catheter insertable into an esophagus near a phrenic nerve. In other examples, electrodes may be positioned on or in an internal or external structure of the body to stimulate one or both phrenic nerves.

Pacing therapy of a phrenic nerve (sometimes referred to as phrenic nerve stimulation therapy or diaphragm pacing), which has many benefits (e.g., preventing or reversing diaphragm muscle-disuse atrophy, maintaining diaphragmatic endurance, facilitating weaning of patients from mechanical ventilation, etc.), may be provided as long-term therapy for patients with conditions unrelated to an airway or lungs (e.g., spinal injury). Patients treated long term with phrenic nerve stimulation may have implanted stimulation leads for improved physiological breathing and mobility of the patient. For patients undergoing phrenic nerve pacing therapy, however, monitoring of tidal volume, flow, nerve signals, and/or other respiratory parameters without invasiveness (other than the implant(s)) is difficult.

Aspects of this disclosure describe systems and methods for acoustic analysis of phrenic nerve stimulation. A stimulation lead, for pacing the phrenic nerve(s), and an acoustic sensor, for detecting sounds or vibrations within the body of the patient, may be placed in the patient. The stimulation lead may be used to stimulate the phrenic nerve(s) to cause movement of the diaphragm (e.g., contraction of the diaphragm), resulting in an associated acoustic signal. The acoustic sensor detects the acoustic signal associated with the diaphragm movement. Other acoustics may be detected by the acoustic sensor, such as heart sounds. The portion of the acoustic signal associated with diaphragm movement is identified for analysis of the phrenic nerve stimulation. Attributes of the portion of the acoustic signal, such as peak frequencies, peak amplitudes, peak durations, etc., may be analyzed to determine resulting tidal volume, smoothness of breathing, intactness of nerve(s), and/or diaphragm muscle atrophy. Stimulation parameters of the phrenic nerve stimulation may be modified or adjusted accordingly. Additionally, the detected acoustics may include a portion attributable to natural breathing efforts of the patient. Pacing may be aligned with detected natural breathing efforts.

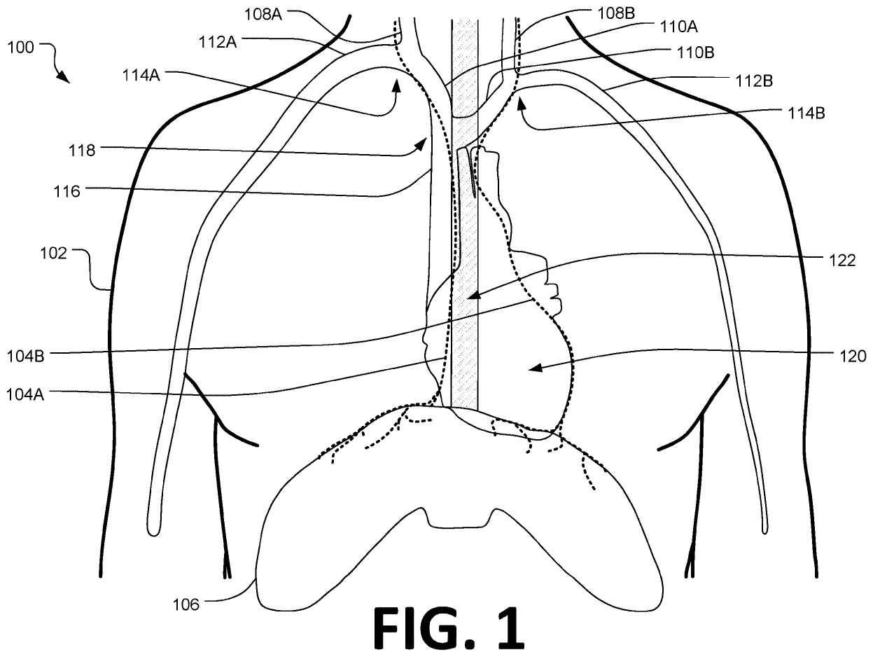

FIG. 1 shows an anatomy of a human patient 100. The body 102 of the patient 100 includes a right phrenic nerve 104A, a left phrenic nerve 104B, a diaphragm 106, a right internal jugular vein 108A, a left internal jugular vein 108B, a right brachiocephalic vein 110A, a left brachiocephalic vein 110B, a right subclavian vein 112A, a left subclavian vein 112B, a right jugular-brachiocephalic junction 114A, a left jugular-brachiocephalic junction 114B, a superior vena cava (SVC) 116, an SVC junction 118, a heart 120, and an esophagus 122 Although not shown, the body 102 of the patient 100 contains other anatomical structures, including a stomach fed by the esophagus 122.

The right phrenic nerve 104A and the left phrenic nerve 104B originate from the spinal cord in the neck region (C3-C5 cervical vertebral region). The right phrenic nerve 104A extends through the body 102 from the right side of the neck region between the right lung and the heart 120 to the right side of the diaphragm 106. The left phrenic nerve 104B extends through the body 102 from the left side of the neck region between the left lung and the heart 120 to the left side of the diaphragm 106. The right phrenic nerve 104A and the left phrenic nerve 104B may each, independently, cause muscle movement of the diaphragm 106 using an electrical signal running down the nerve. The phrenic nerve is “stimulated” when the phrenic nerve sends an electrical signal to the diaphragm 106. When the phrenic nerve is “effectively stimulated” or “captured,” one or more muscles of the diaphragm 106 move (such as stiffening or contracting) due to the electrical signal sent from the phrenic nerve. The electrical signal to stimulate the phrenic nerve may originate naturally from the brain or may be provided artificially (e.g., by a nearby electrical current, and resulting voltages, from electrodes associated with a stimulation device). For example, the right phrenic nerve 104A and/or the left phrenic nerve 104B may be artificially stimulated by voltages resulting from an electrical current between one or more electrodes on a stimulation device (such as an intravenous stimulation device or an esophageal stimulation device). Examples of stimulating one or both phrenic nerves via an intravenous stimulation lead are provided in U.S. patent application Ser. No. 17/039,115, titled “Intravenous Phrenic Nerve Stimulation Lead,” and filed on Sep. 30, 2020, which is incorporated herein by reference in its entirety. Additionally, examples of stimulating one or both phrenic nerves via an esophageal balloon catheter are provided in U.S. Patent Application Ser. No. 63/241,747, titled “Phrenic Nerve Stimulation with Mechanical Ventilation,” and filed on Sep. 8, 2021, which is incorporated herein by reference in its entirety. As voltages are developed near the phrenic nerve, transmission fibers of the phrenic nerve are excited to create movement of muscles in the diaphragm. For the induced voltages to effectively stimulate the phrenic nerve, the one or more electrodes carrying the electrical current should be placed near, adjacent, or proximate the right phrenic nerve 104A and/or the left phrenic nerve 104B.

The electrical signal sent to the diaphragm 106 from the right phrenic nerve 104A and/or the left phrenic nerve 104B causes movement of one or more muscles of the diaphragm 106. Muscle movement of the diaphragm may cause expansion of the lungs of the patient. For example, stimulation of the right phrenic nerve 104A causes one or more muscles on a right portion of the diaphragm 106 to move, thereby causing expansion of one or both lungs of the patient 100. In another example, stimulation of the left phrenic nerve 104B causes one or more muscles on a left portion of the diaphragm 106 to move, thereby causing expansion of one or both lungs of the patient 100. Stimulation of only one of the phrenic nerves (i.e., right phrenic nerve 104A or left phrenic nerve 104B) may cause muscle movement on both sides of the diaphragm, due to the collective nature of the diaphragm muscles (e.g., stimulating muscles on one side will also move some of the muscles on the other side). For some patients, stimulation of only one of the phrenic nerves is sufficient, while other patients may require or desire stimulation of both phrenic nerves. In some instances, the integrity of a phrenic nerve (e.g., a damaged phrenic nerve) may be assessed for viability of phrenic nerve stimulation of the diaphragm. Examples of stimulating one or both phrenic nerves to obtain desirable movement of the diaphragm are provided in U.S. patent application Ser. No. 16/888,960, titled “Achieving Smooth Breathing by Modified Bilateral Phrenic Nerve Pacing,” and filed on Jun. 1, 2020, and U.S. Patent Application Ser. No. 63/391,430, titled “Phrenic Nerve Integrity and Ramped-Up Burst,” and filed Jul. 22, 2022, the entireties of which are incorporated herein by reference in their entireties.

In an example, a phrenic nerve (e.g., the right phrenic nerve 104A and/or left phrenic nerve 104B) may be stimulated by voltages induced by a current between two or more electrodes. The electrodes may be positioned inside of the body of a patient at a location where a phrenic nerve is near the resulting induced voltages. In an example, the electrodes may be coupled to a stimulation device to facilitate introduction into the body. For instance, electrodes may be placed intravenously (e.g., via an intravenous stimulation lead) adjacent to the phrenic nerve, or esophagealy (e.g., via a stimulation device in an esophagus 122 of the patient 100) adjacent to the phrenic nerve or directly on the diaphragm.

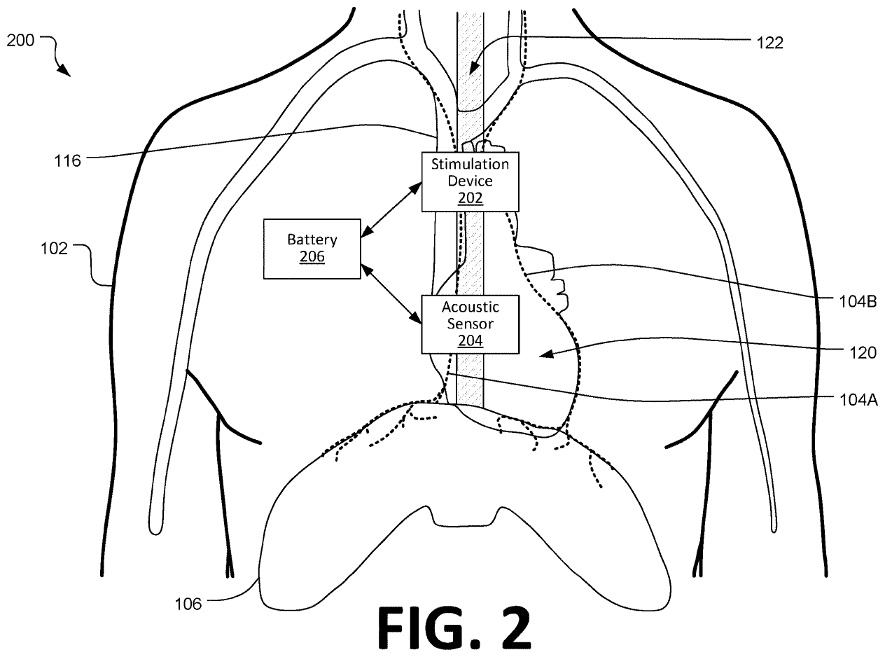

FIG. 2 shows a stimulation device 202 and an acoustic sensor 204 placed in the body 102 of the patient 200 of FIG. 1. The stimulation device 202 includes at least one set of electrodes capable of stimulating a nerve (e.g., right phrenic nerve 104A or left phrenic nerve 104B). The stimulation device 202 may be used to stimulate one or both phrenic nerves for pacing of muscles of the diaphragm 106. Placement of the stimulation device 202 may be along different depths of vessel(s) in the body 102 (e.g., a blood vessel or an esophagus 122) proximate one or both phrenic nerves 104A, 104B or directly on the diaphragm. In an example where the stimulation device 202 is placed in a blood vessel (e.g., intravenously), the stimulation device 202 may be a lead placed via a catheter inside any blood vessel in the body 102 that runs proximate a phrenic nerve (e.g., the right internal jugular vein 108A, the left internal jugular vein 108B, the right brachiocephalic vein 110A, the left brachiocephalic vein 110B, the right subclavian vein 112A, the left subclavian vein 112B, the right jugular-brachiocephalic junction 114A, the left jugular-brachiocephalic junction 114B, SVC 116, SVC junction 118, inferior vena cava, carotid arteries, subclavian arteries, aorta, etc.). Alternatively, in an example where the stimulation device 202 is placed esophagealy, electrodes may be coupled to an inflatable balloon of a balloon catheter securable in an esophagus 122. The specific placement of the stimulation device 202 shown in FIG. 2 is shown as an example, however, placement of the stimulation device 202 may be at any other location proximal to one or both phrenic nerves 104A, 104B.

The stimulation device 202 may include features or components to secure the stimulation device 202 inside the body 102. For example, the stimulation device 202 may include one or more deformable segments and one or more elongate segments. Electrodes may be placed on deformable segments and/or elongated segments of the stimulation device 202. In another example, the stimulation device 202 may include an inflatable balloon. Electrodes may be placed on, about, or proximate the inflatable balloon.

The stimulation device 202 is capable of providing voltage to one or more electrodes coupled to the stimulation device 202 from a power source (e.g., a battery 206). The battery 206 may be securable to the body 102 of the patient, such as via implant, inserted subcutaneously or percutaneously, secured to skin, or securable or movable with the body 102 of the patient. The battery 206 may be shared with other devices or sensors, such as an acoustic sensor 204 in the body 102. The battery 206 may be removable, replaceable, and/or rechargeable (e.g., with or without removing the battery 206 from the body 102).

Attributes (e.g., voltage, frequency, pulse width, duty cycle, etc.) of each electrode of the stimulation device 202 may be individually addressable and controllable by a controller (such as by circuitry on a PCB). Delivery of the attributes to one or more electrodes may be referred to herein as settings for phrenic nerve stimulation (PNS). In an example, a clinician may control the nerve pacing (e.g., PNS settings) of the stimulation device 202 via the controller, and observe the resultant ventilatory efforts of the patient at a device external to the body 102 of the patient (e.g., at a ventilator or other external device with a user interface). The controller may be a component of a ventilator or may be a separate device. The controller may be shared between one or more devices or sensors associated with the patient (e.g., a shared controller between a stimulation device 202 and an acoustic sensor 204). In an example where the controller is integrated into a ventilator, the power source may be provided by the ventilator (e.g., instead of or in addition to a battery 206). In an example where the controller is a separate device, the controller may be associated with a display and user interface to allow viewing or selecting of stimulation pulse attributes.

The voltages produced by the electrodes of the stimulation device 202 may be extended. For example, an external pad may be placed on or near the skin of the patient, external to the patient. The external pad may include one or more electrodes. The external pad may be moved relative to the body 102 of the patient 100 and/or relative to the stimulation device 202 to provide more or less stimulation of a phrenic nerve of a patient.

An acoustic sensor 204 may be placed in the body 102 (e.g., implanted) or on the body 102 (e.g., placed on the skin) of the patient. The acoustic sensor may be any sensor capable of detecting sound (e.g., sound or vibrations caused by movement of a valve, muscle, organ, vessel, gas, liquid, joint, or any other part or component of the body 102), such as a piezoelectric crystal, leads, microphone, accelerometer, vibration sensor, etc. The acoustic sensor 204 may be a component of an implantable cardioverter-defibrillator (ICD), pacemaker, or other heart health device. The acoustic sensor 204 may also be part of another medical device, such as a subcutaneously implanted medical device. Alternatively, the acoustic sensor 204 may be standalone and positioned proximate the diaphragm 106. In an example, the acoustic sensor 204 is capable of detecting heart sounds and sounds caused by movement of the diaphragm. The acoustic sensor 204 may be placed proximate the heart 120, such as mounted under the xiphoid process of the patient (e.g., mounting of the acoustic sensor 204 at an inner wall of the thoracic cavity), on a portion of the heart 120, in the chest with leads extending towards the heart 120, under the skin near an armpit (e. g, axillary) with an electrode along the breastbone, or otherwise positioned near the heart 120. The acoustic sensor 204 may include a securing mechanism to secure the acoustic sensor 204 in or on the body 102. In an example where the acoustic sensor 204 is inside of the body 102, the securing mechanism may be configured to secure the acoustic sensor to patient tissue, such as via anchors, adhesive, sutures, pins, screws, suction, etc.

As used herein, the term heart sound refers to a feature of a heart sound signal that may be associated with acoustic vibrations of a patient's heart, heart valves, and/or flow of blood through heart valves, veins, or arteries. There may be multiple heart sounds for any given cardiac cycle or heart beat (e.g., an S1, S2, S3 and/or S4 heart sound). The first heart sound of a heart beat is referred to as “S1” and is caused by vibrations resulting from closure of the atrioventricular (AV) valves (e.g., closing of the mitral valve and/or tricuspid valve). The second heart sound is referred to as “S2” and is caused by vibrations resulting from closure of the semilunar valves (e.g., the pulmonary and/or aortic valves). The third and fourth heart sounds are referred to as “S3” and “S4,” respectively, and are caused by vibrations resulting from filling of ventricles with blood. S3 is associated with rapid filling of the ventricles (e.g., which can occur when the ventricular wall is not relaxed when a large volume of blood flows into the stiffened ventricle from the atrium). S4 is caused by blood rapidly filling into the ventricles from the atria (e.g., which can occur during atrial contraction, and may be referred to by some as “atrial kick”).

As described above the acoustic sensor 204 detects sounds other than heart sounds. For example, the acoustic sensor 204 detects sounds (e.g., vibrations) associated with breathing. The acoustic sensor 204 may be capable of detecting sounds resulting from movement of the diaphragm (e.g., due to spontaneous breathing of the patient and/or cause by phrenic nerve stimulation). Additionally or alternatively, the acoustic sensor 204 may be capable of detecting sounds caused by movement and/or function of the lungs (e.g., respiratory rate and/or conditions such as coughing, rales, rhonchi, stridor, or wheezing, etc.).

Other devices and/or sensors may be in, on, or about the body 102 of the patient 200. For example, devices or sensors that may assist in identification or differentiation of sounds and/or vibrations may be used. For instance, sensors and/or electrodes for an electrogram (EGM) and/or an electrocardiogram (ECG) (not shown) and/or other heartrate monitors may be used to better identify heart sounds. Additionally or alternatively, sensors for detecting a patient effort and/or lung or diaphragm movement (not shown) may be used to better identify sounds associated with diaphragm movement.

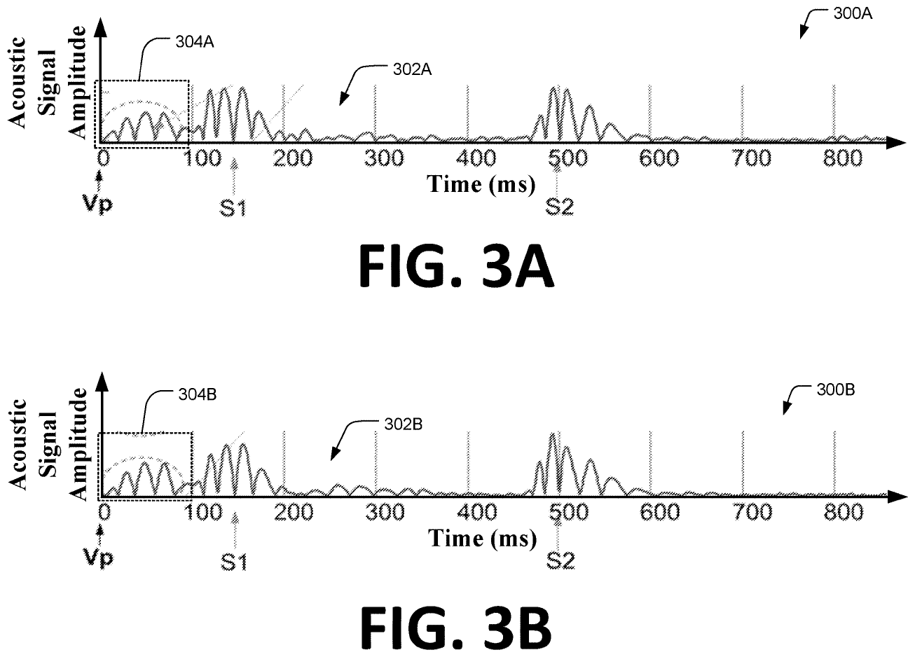

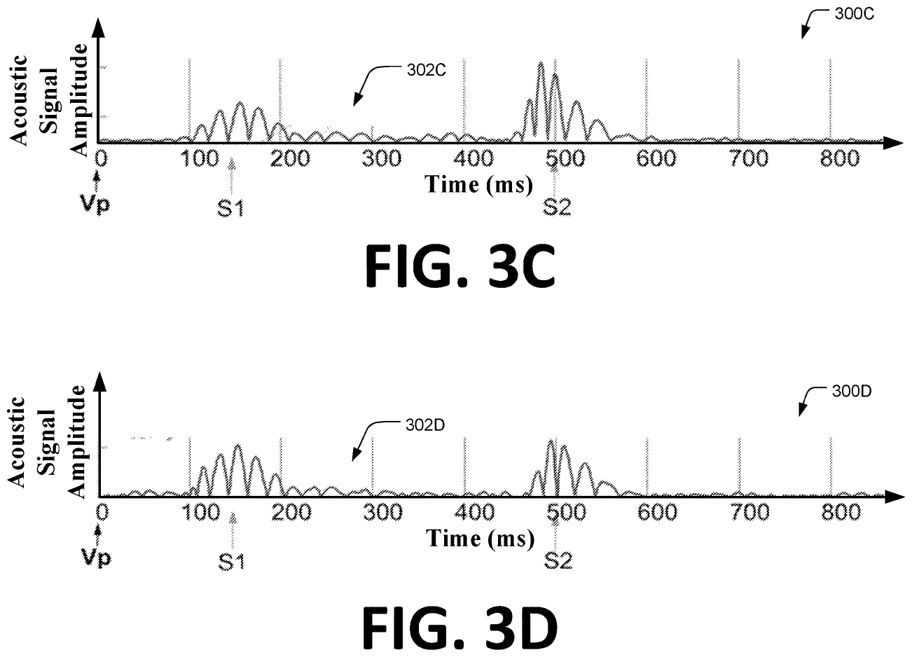

FIGS. 3A-3D show graphical representations 300A-300D of acoustic signals 302A-302D. The acoustic signals 302A-302D are shown as an acoustic signal amplitude versus time. By way of example, each of the acoustic signals 302A-302D shows an average of the acoustic signal detected (e.g., by an acoustic sensor) over multiple breaths for a cardiac cycle (e.g., including S1 and S2 heart sounds in the acoustic signal) as acquired via ICD and filtered to reduce noise. In the examples of acoustic signals 302A-302D shown in FIGS. 3A-3D, phrenic nerve stimulation (PNS) is delivered at PNS time vₚ (e.g., at t=0, prior to S1). Although the PNS time, vₚ, is prior to and proximate S1 in the cardiac cycle of FIGS. 3A-3D, the PNS time vₚ, may occur at a different time in relation to the cardiac cycle or may vary between cardiac cycles. For example, a frequency of PNS may be approximately every 4-8 seconds and a cardiac cycle may be approximately every 1-2 seconds.

FIGS. 3A-3B show example acoustic signals 302A, 302B that include a PNS contribution 304A, 304B caused by movement of the diaphragm as a result of delivering PNS at PNS time vₚ. Although the PNS contributions 304A, 304B shown in FIGS. 3A-3B occur in the 100 ms immediately following delivery of PNS at the PNS time, a contraction or movement of the diaphragm in response to PNS may vary from patient to patient (e.g., from 0-400 ms after PNS is delivered). For example, a first patient's diaphragm may contract in the first 100 ms post-PNS and a second patient's diaphragm may contract after the first 100 ms. Comparing the acoustic signal 302A in FIG. 3A with the acoustic signal 302B in FIG. 3B, the acoustic signal 302A in FIG. 3A, indicates less responsiveness of the diaphragm than the acoustic signal 302B in FIG. 3B. The peak amplitude of the PNS contribution 304A in FIG. 3A is less than the peak amplitude of the PNS artifact 304B in FIG. 3B, and the amplitude of the diaphragmatic contributions may vary over time within a patient, with respect to the acquired heart sounds, and also varies from patient to patient.

FIG. 3C shows an example acoustic signal 302C that does not include a PNS contribution following the PNS time, vₚ, of PNS delivery. Heart sounds (e.g., S1 and S2) are still present in the acoustic signal 302C, indicating that the acoustic sensor is operational. The PNS contribution may not be present in acoustic signal 302C because the corresponding PNS signal did not effectively stimulate or capture the diaphragm. FIG. 3D shows an example acoustic signal 302D that includes some noise during a time period of an expected PNS artifact (e.g., in the first 100 ms after the PNS time vₚ of PNS delivery). The noise in this time period may be attributable to some natural (e.g., spontaneous) breathing contribution by the patient, other muscle movement, and/or other vibrations detected by the acoustic sensor. Such noise may be filtered from the acoustic signal to reduce the likelihood of being mistaken for a PNS contribution.

In some examples, one or more acoustic artifacts of the PNS may occasionally occur synchronously with one of the heart sounds S1 or S2, where the heart sounds may obscure the PNS acoustic artifacts. As described, averaging the acquired acoustic signal over multiple breaths, such as in the waveforms of FIG. 3, produces a waveform that may emphasize PNS artifacts that are distinct from heart sounds S1 and S2.

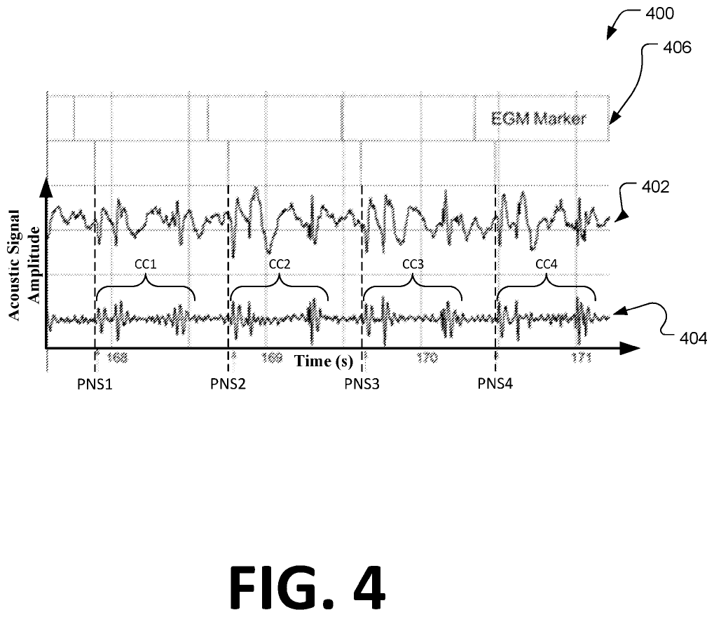

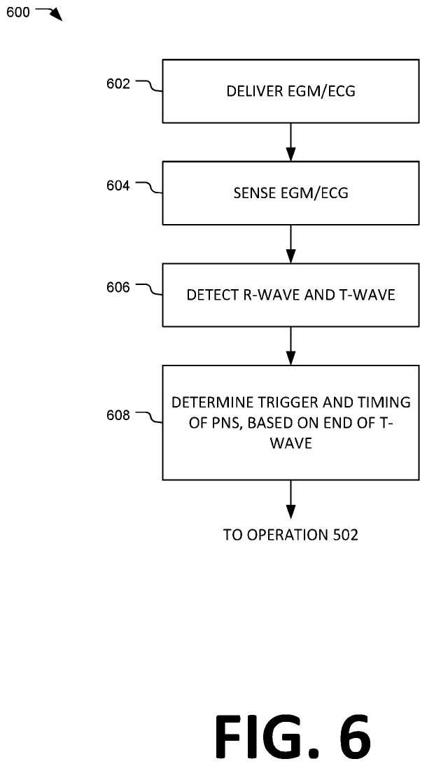

FIG. 4 shows another graphical representation 400 of acoustic signals 402, 404. The graphical representation 400 includes an unfiltered acoustic signal 402 and a filtered acoustic signal 404 plotted as acoustic signal amplitude versus time. An electrogram (EGM) marker 406 is also shown versus time in the graphical representation 400, indicating timing of cardiac cycles. Like FIGS. 3A-3D, by means of example, the delivery of PNS is timed at the beginning of each cardiac cycle. (Additional details of the timing of PNS delivery in relation to EGM markers and/or other aspects of an associated ECG are provided in FIG. 6 below). As shown in FIG. 4, a first nerve pace PNS1 is timed with a first cardiac cycle CC1, a second nerve pace PNS2 is timed with a second cardiac cycle CC2, a third nerve pace PNS3 is timed with a third cardiac cycle CC3, and a fourth nerve pace PNS4 is timed with a fourth cardiac cycle CC4. As otherwise described herein, pacing of the phrenic nerve(s) may be delivered at a different frequency than the frequency of cardiac cycles (e.g., PNS may be delivered every 4-8 seconds and cardiac cycles may occur every 1-2 seconds).

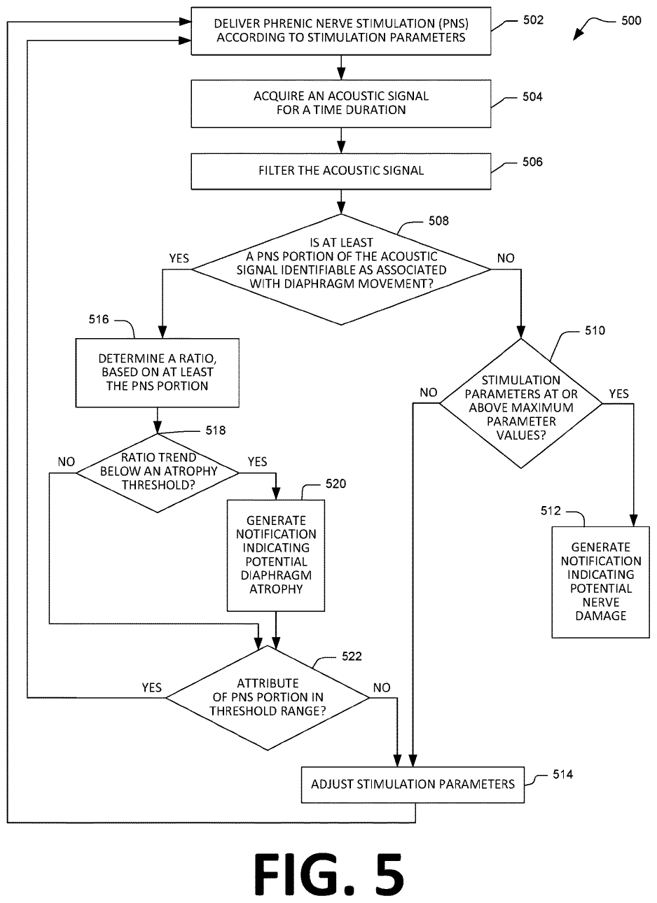

FIG. 5 shows a method 500 for identifying diaphragm movement based on an acoustic signal. In some examples, the method 500 may be performed by a ventilator, or components thereof, that is coupled to an acoustic sensor and/or a PNS system. In other examples, the method 500 is performed a separate controller, coupled to the acoustic sensor and/or the PNS system, that includes a processor and memory for processing and storing the signals discussed herein.

At operation 502, phrenic nerve stimulation (PNS) is delivered. One or both phrenic nerves of a patient may be stimulated. The stimulation may be delivered via electrodes of a stimulation device (e.g., stimulation devices 202) PNS may be delivered according to one or more stimulation parameters, such as electrode pair selection, pulse width, frequency (e.g., 40 Hz), amplitude/current, delivered charge, burst length, burst ramp-up, etc. Stimulation of the diaphragm may be based on the parameters of PNS and/or the presence of nerve damage. For example, modifying one or more parameters of PNS may increase or decrease diaphragm movement in response to the PNS. Alternatively, a change in PNS parameters may not result in a change of diaphragm movement if the phrenic nerve(s) are damaged (e.g., loss of communication between the nerves and the diaphragm, such as along the nerve or at the nerve-muscle connection). At the first performance of operation 502, the PNS may be provided at a relatively low level (e.g., low amplitude) to prevent unintentional overstimulation of the diaphragm, which may cause a hiccup or other similar effect. As method 500 iteratively repeats, the PNS level (e.g., amplitude) may be increased, as discussed further herein.

At operation 504, an acoustic signal is acquired for a time duration. As described above, the acoustic signal results from vibrations caused by movement of muscles, tendons, fluids, gases, etc. The acoustic signal may be acquired or detected by the acoustic sensor and stored in memory for the analysis and processing described herein. For instance, during PNS, an acoustic signal may include a first portion attributable to movement of the diaphragm (e.g., as caused by natural breathing and/or movement resulting from PNS) and a second portion attributable to movement of the heart (e.g., heartbeats or heart sounds). Depending on the respiratory rate, reaction time of the diaphragm (e.g., the time delay between PNS and movement of the diaphragm caused by the PNS), and heartrate of the patient, the first portion and the second portion of the acoustic signal may overlap.

The time duration that the acoustic signal is detected may vary as required or desired. For example, in in an initial phase, the acoustic signal may be captured for a longer period of time to allow for additional data to be analyzed to detect or identify the PNS contributions within the acoustic signal. The time duration may range from 2 to 200 seconds. In an instance, the time duration may be 80-120 seconds (e.g., 100 seconds). After a reaction time of the diaphragm is known for the patient, the time duration may be 2-10 seconds (e.g., 3 seconds). Long time durations allow for more heartbeats to be more accurately identified and detected (e.g., for which heartbeats occur at predictable intervals/frequencies according to a pattern approximately every second). Accurate identification of heart sounds may allow for more accurate distinguishing of heart sounds from acoustics attributable to movement of the diaphragm (e.g., at predictable intervals/frequencies according to a pattern approximately every 2-3 seconds). In some examples, where possible, an acoustic signal is captured without any PNS being performed to provide an acoustic signal that does not have any PNS contribution. Such a PNS-free acoustic signal may be used as a baseline for filtering future signals that include both cardiac and diaphragmatic contributions.

At operation 506, the acoustic signal is filtered. Filtering of the acoustic signal may remove or reduce noise in the acoustic signal. Noise may be any vibrations or sounds not associated with diaphragm movement and/or cardiac sounds. Filtering of the acoustic signal may be performed by a processor and/or analog filters. The filter may filter out certain frequencies and/or amplitudes of the acoustic signal. In an example, the filter is a band-pass filter. Multiple filters may be applied, such as a high band-pass filter and/or low band-pass filter. Other non-linear filtering techniques may also be applied in the time and/or frequency domains.

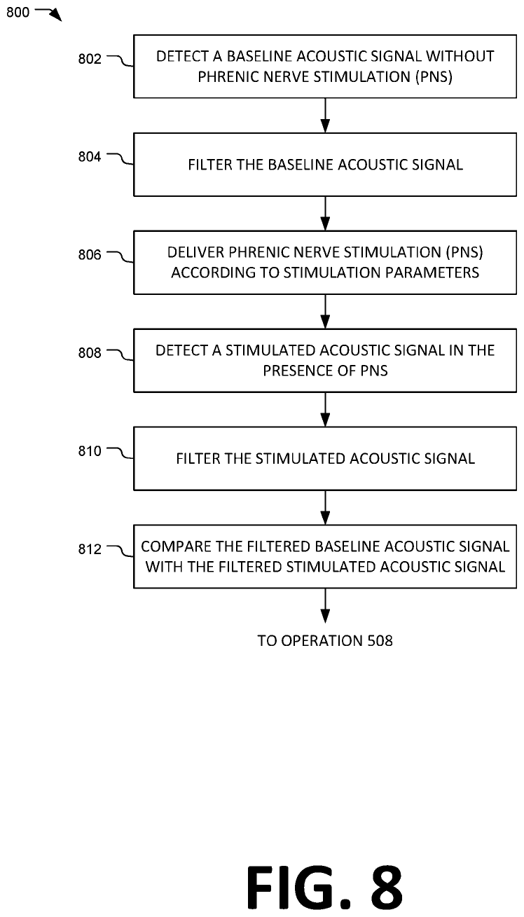

At determination 508, a determination is made as to if at least a PNS portion of the acoustic signal, associated with diaphragm movement, is identifiable. As discussed herein, heart sounds may be distinguished from sounds associated with diaphragm movement. The portion of the acoustic signal associated with diaphragm movement may be identified using pattern recognition, machine learning, comparison with other acoustic signals of the patient, and/or known patient parameters (e.g., stimulation parameters, heart rate, respiratory rate, reaction time of the diaphragm, etc.). In an example, the PNS portion of the acoustic signal may be identified by pausing PNS temporarily to detect a baseline acoustic signal for comparison with the acoustic signal detected during PNS. An example of this comparison is further described at least with respect to FIG. 8. The comparison may result in an acoustic signal without heart sounds or a signal only attributable with diaphragm movement. As described herein, the PNS portion may be a portion of the acoustic signal attributable to any movement of the diaphragm (e.g., natural movement resulting from spontaneous breathing of the patient and artificial movement resulting from PNS and/or ventilation). The PNS portion may also be a portion of the acoustic signal attributable to artificial movement of the diaphragm.

Additionally or alternatively, a heart sounds portion of the acoustic signal may be identified at determination 508. Identification of the heart sounds may also be used to determine if a PNS portion of the acoustic signal is identifiable (e.g., if any diaphragm movement is detected). Identification of heart sounds in the acoustic signal may be based on pattern recognition, machine learning, markers, and/or a comparison of signals (e.g., method 800 of FIG. 8). For example, frequencies, amplitudes, and/or shapes of the acoustic signal may be used to identify portions of the acoustic signal associated with cardiac sounds. For instance, amplitudes recurring every 1-2 seconds, peaks with higher amplitudes, sharper slopes, and/or peaks aligning with an EGM/ECG marker, may be associated with heart sounds, rather than noise or diaphragm movement. Other identification of acoustics associated with cardiac sounds is described herein.

If, at determination 508, no portion of the acoustic signal is identifiable as associated with diaphragm movement, flow proceeds “NO” to determination 510. At determination 510, a determination is made as to whether the stimulation parameters of PNS are at or above maximum parameter values. Maximum parameters values may be preset or configured for the patient to prevent or reduce injury from PNS. Maximum parameter values may include a maximum frequency, amplitude, pulse width, or ramp up of PNS. The maximum parameter values may be based on patient information, such as weight, predicted body weight (PBW), sex, age, respiratory conditions, health conditions, etc. For example, a patient with a higher PBW may have a higher maximum parameter value for amplitude than a patient with a lower PBW. In an example, maximum parameters values may be a multiple of at least one of the initial stimulation parameters. For instance, for an initial voltage V1 for PNS at operation 502, an adjustment up to a maximum parameter value of three times V1 (e.g., V3=3*V1) may be permissible before stimulation parameters are determined to be at or above maximum parameter values. The maximum parameter values may be set for patient safety during PNS.

If, at determination 510, the stimulation parameters are at or above the maximum parameter values, flow proceeds “YES” to operation 512. At operation 512, a notification is generated. The notification may indicate that the patient potentially has nerve damage. In method 500, prior to operation 512, there is no portion of the acoustic signal identified as associated with diaphragm movement and the stimulation parameters according to which PNS is delivered to the patient are at or exceeding maximum values. Assuming that a stimulation device providing the PNS is properly placed to stimulate the nerve(s), the PNS is not causing any detectable movement of the diaphragm identifiable in the acoustic signal. This indicates potential damage at or along the nerve(s) and/or nerve-muscle connection as damage may prevent or restrict PNS from stimulating the diaphragm (e.g., the nerve(s) are not intact). Damage of the nerve(s) may present prior to the patient entering care and/or may occur over the course of PNS due to some neurodegenerative diseases. The notification of nerve damage may be presented as an alert to a clinician for further evaluation. In an example, the stimulation device may be movable along the nerve(s) or otherwise evaluated for dysfunctionality. In another example, the notification may indicate that no diaphragm movement is detected and/or that PNS stimulation parameters are at a maximum.

If, alternatively, at determination 510, the stimulation parameters are below the maximum parameter values, flow proceeds “NO” to operation 514. At operation 514, the stimulation parameters are adjusted. Along this flow path of method 500 (e.g., from determination 510 to operation 512 to operation 514), the stimulation parameters may be adjusted to increase stimulation of the nerve(s). For example, a frequency, amplitude, pulse width, delivered charge, cycling ratio, or ramp up of the PNS may be increased. Additionally or alternatively, more electrodes or different pairs of electrodes of the stimulation device delivering PNS may be selected for delivery of stimulation. Method 500 then returns to operation 502 where PNS is delivered with the adjusted stimulation parameters and method 500 is repeated.

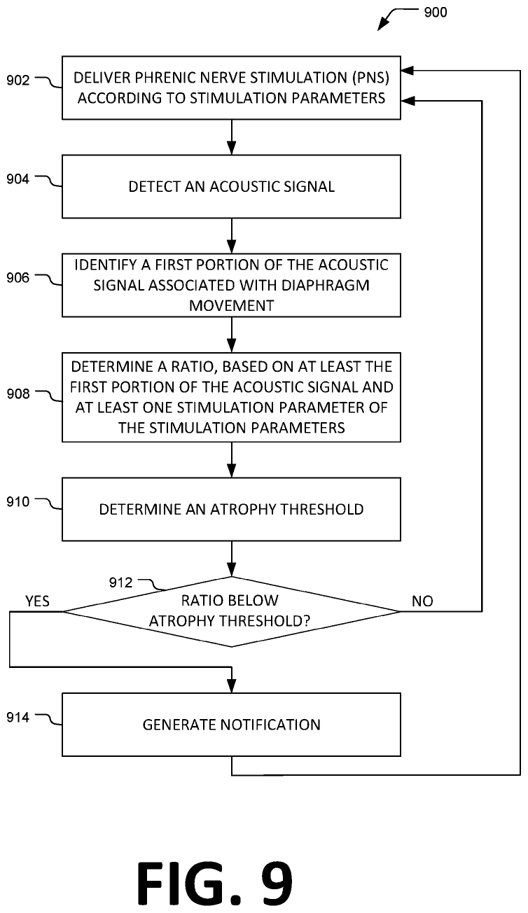

If, alternatively, at determination 508, at least a portion of the acoustic signal is identifiable as associated with diaphragm movement, flow proceeds “YES” to operation 516 and determination 518. Operations 516-520 evaluate the diaphragm for atrophy based on a ratio. Use of a ratio to evaluate diaphragm atrophy is further described at least with respect to FIG. 9.

At operation 516, a ratio between acoustic properties of the PNS contribution in the acoustic signal and the electrical properties of the PNS is determined. One component of the ratio may be based on an acoustic attribute of the PNS contribution in the acoustic signal, such as average amplitude, peak amplitude, signal energy, shape of peak, width or duration of peak, slope of peak, area under the curve of the PNS contribution in the acoustic signal, or any other measurement or visual characteristic of the acoustic signal. The acoustic attribute of the PNS contribution may be correlated with measures of breathing effectiveness, such as tidal volume, smoothness of breathing, or other measures. For example, the amplitude of the PNS contribution of the acoustic signal may be correlated with tidal volume.

A second component of the ratio may also be based on one or more stimulation parameters. The ratio may relate an acoustic attribute of the PNS contribution with an associated stimulation parameter, for example, amplitude and voltage, slope of peak and ramp up, duration of peak and pulse width, etc. For instance, the ratio may relate an amplitude for a PNS contribution of the acoustic signal (e.g., a peak amplitude or average amplitude for a breath) with a voltage of PNS.

This ratio is indicative of how strongly the diaphragm responds to the PNS at particular electrical energy levels. For example, the ratio provides a comparison of the acoustic energy in the PNS contribution of the acoustic signal and the electrical energy in the PNS signal. Thus, the ratio determined in operation 516 may be referred to as an acoustic-to-electrical PNS ratio. This acoustic-to-electrical PNS ratio may change in a manner that indicates additional electrical energy is required to generate the same acoustic energy, resulting in a decrease in the numeric value of the PNS ratio. Such a change in the ratio may indicate that the diaphragm is beginning to atrophy. Accordingly, the trend in the acoustic-to-electrical PNS ratio may be monitored to determine physiological changes in the diaphragm.

Alternatively, the PNS ratio may be determined as the ratio of electrical properties of the PNS to the acoustic properties of the PNS. For example, the PNS ratio may provide a comparison of the electrical energy of the PNS signal to the acoustic energy in the PNS contribution of the acoustic signal. Thus, the ratio determined in operation 516 may be referred to as an electrical-to-acoustic PNS ratio. In such examples, the need for additional electrical energy to generate the same acoustic energy may result in an increase in the electrical-to-acoustic PNS ratio, as opposed to a decrease in the acoustic-to-electrical PNS ratio. To facilitate description, as used herein, operations that relate to the PNS ratio will be in reference to the acoustic-to-electrical PNS ratio. Accordingly, any corresponding thresholds referred to herein will likewise be in reference to the acoustic-to-electrical PNS ratio.

At determination 518, a determination is made as to if a trend ratio is below an atrophy threshold. For instance, a change in the ratio over a period of time is compared to an atrophy threshold. A ratio trend falling below the atrophy threshold may be associated with diaphragm atrophy. The ratio trend may be determined based on a moving window of the ratio, such as a set quantity of breaths, heartbeats, and/or time preceding the current ratio (e.g., the ratio for the last identified PNS portion). The atrophy threshold may be percentage change over the moving window or an adjustment of the average over the moving window. For example, a percentage change (e.g., 50%, 60%, 70%, 75%, 80%, etc.) may be set as the atrophy threshold. The atrophy threshold may be based on sets of clinical data where diaphragm atrophy has been identified.

If, at determination 518, the ratio trend is below the atrophy threshold, flow proceeds “YES” to operation 520. At operation 520, a notification indicating potential atrophy is generated. The notification may indicate that the patient has diaphragm atrophy or potentially has diaphragm atrophy. Atrophy of the diaphragm may be possible over time if the diaphragm is over or under exercised. The notification may indicate other potential therapy methods available for the patient or other steps to reduce the atrophying of the diaphragm. If, alternatively, at determination 518, the ratio is at or above the atrophy threshold, flow proceeds “NO” to determination 522.

At determination 522, a determination is made as to if an attribute of the PNS contribution in the acoustic signal of the PNS portion is in a threshold range that indicates adequate or desired stimulation capture of the diaphragm. For instance, the attributes of the PNS contribution may indicate how strongly the diaphragm contracted in response to the PNS and how for how long the contraction occurred. Such indications may be used to assess the effectiveness of the PNS, including in some cases an estimate of the tidal volume that was inhaled as a result of the diaphragm contraction.

A peak amplitude and/or duration of the PNS contribution may be used as an attribute of the PNS contribution of the acoustic signal for analysis. As an example, a peak amplitude below a lower threshold of the threshold range may indicate that the movement of the diaphragm is not enough to result in a desirable tidal volume of the patient (e.g., tidal volume resulting from PNS being too low). A peak amplitude above an upper threshold of the threshold range may indicate that movement of the diaphragm is too sharp (e.g., breathing is not smooth and/or a hiccup is experienced). A peak amplitude within the threshold range may indicate that the movement of the diaphragm is associated with desirable or expected contraction of the diaphragm (e.g., movement of the diaphragm resulting in smooth breathing and sufficient tidal volume) based on the delivered PNS.

The threshold range may be determined or set from patient to patient. The threshold range may be determined in a calibration procedure (e.g., the threshold range may be calibrated for each patient). The threshold range may vary from patient to patient based on patient parameters, the acoustic sensor, placement of the acoustic sensor relative to the heart and/or diaphragm of the patient, etc. In an example, the threshold range may be associated with an acoustic signal detected at different tidal volumes delivered to a patient. For instance, stimulation parameters may be adjusted until a desired tidal volume is delivered to a patient. A portion of the resulting acoustic signal may be identified as associated with movement of the diaphragm for the desired tidal volume. That portion of the acoustic signal may be used to determine a threshold range about the peak amplitude associated with the desired tidal volume. Alternatively, the threshold range for the peak amplitude may be set based on patient information and/or position of the acoustic sensor relative to the diaphragm. For example, an acoustic sensor placed further from the diaphragm may have a threshold range lower than an acoustic sensor placed closer to the diaphragm. As another alternative, the threshold range may be determined based on a ratio of the PNS portion with another portion of the acoustic signal (e.g., the whole acoustic signal, a portion associated with heart sounds, etc.). For example, the threshold range may be based on a ratio of peak amplitude of a PNS portion to a peak amplitude of cardiac sounds. As another example, the threshold range may be based on a ratio of an amplitude of a PNS portion with an amplitude of the total acoustic signal or an amplitude of cardiac sounds detected near the PNS portion.

Although the threshold range is described above with respect to amplitude of the acoustic signal, or portion of the acoustic signal, the threshold range may additionally or alternatively consider other attributes of the acoustic signal. Attributes of the acoustic signal that may have a threshold range include average amplitude, peak amplitude, signal energy, shape of peak, width or duration of peak, slope of peak, or any other measurement or visual characteristic of the acoustic signal. The threshold range may include multiple attributes of the acoustic signal. For example, the threshold range may be a peak amplitude and a duration (e.g., width) of the peak amplitude for a PNS portion.

If, at determination 522, the attribute of the acoustic signal of the PNS contribution is in the threshold range, flow proceeds “YES” back to operation 502. Operations 502-522 may repeat as required or desired with the previous stimulation parameters for PNC. For example, while an attribute of the PNS portion of the acoustic signal is within a threshold range, PNS may continue to be delivered according to the current stimulation parameters.

If, alternatively, at determination 522, the attribute of the acoustic signal of the PNS contribution is outside of the threshold range, flow proceeds “NO” to operation 514. At operation 514, the stimulation parameters are adjusted. Along this flow path of method 500 (e.g., from determination 508 to determination 522 to operation 514), the stimulation parameters may be adjusted to increase stimulation of the nerve(s) if the acoustic attribute of the PNS contribution is below the threshold range (e.g., the patient is not achieving a desired tidal volume) or adjusted to decrease the stimulation of the nerve(s) if the attribute of the PNS portion is above the threshold range (e.g., patient is experiencing hiccups). For example, a frequency, amplitude, pulse width, delivered charge, cycling ratio, or ramp up of the PNS may be adjusted. Additionally or alternatively, more electrodes or different pairs of electrodes of the stimulation device delivering PNS may be selected for delivery of stimulation.

Operations 502-522 may repeat as required or desired. For example, while no diaphragm movement is detected at determination 508, stimulation parameters may be adjusted or increased until the stimulation parameters meet or exceed the maximum parameter values (e.g., determination 508 to determination 510 to operation 514). In another example, if diaphragm movement is detected, but an attribute of the PNS portion of the acoustic signal is outside of a threshold range at determination 522, the stimulation parameters may be adjusted or increased (e.g., flowing “NO” at determination 522) until the attribute of the PNS portion falls within the threshold range (e.g., flowing “YES” at determination 522).

This method 500 allows for periodic evaluation of diaphragm movement, via acoustic analysis, to allow for information to be delivered to a clinician during a closed-loop control of PNS. If diaphragm atrophy or nerve damage is suspected, the clinician may be notified to allow for intervention (e.g., at operations 512, 520). Clinician intervention may include evaluation of the stimulation device, the patient, and/or available respiratory therapies. The stimulation device may be repositioned (e.g., in a different depth of a vein or in the esophagus), replaced (e.g., with a stimulation device or a same or different type), and/or repaired.

FIG. 6 shows a method 600 for timing phrenic nerve stimulation, based on the cardiac cycle. At operation 602, an electrogram (EGM) and/or an electrocardiogram (ECG) is delivered. Delivery may be performed by a heart health device (e.g., ICD). The heart health device may include an acoustic sensor (e.g., as described to carry out method 500 in FIG. 5). At operation 604, the EGM and/or ECG is sensed. Sensing of the EGM and/or ECG may be performed by the same or different device than that delivering the EGM and/or ECG at operation 602. Based on sensing the EGM and/or ECG, an R-wave and a T-wave associated with a cardiac cycle are detected at operation 608. Detection of the T-wave may also include detecting an end of the T-wave.

At operation 608, a trigger and timing of phrenic nerve stimulation (PNS) are determined, based on an end of the T-wave. For example, pacing of the phrenic nerve(s) may be delayed (e.g., timed) for a set delay after the end of the T-wave (e.g., trigger), such as 100-500 ms after the end of the T-wave. In a typical cardiac cycle, the heart is resting for a rest duration after the end of the T-wave. When the heart is resting, little to no acoustics are originating from the heart and the heart is not moving. Thus, timing of PNS after the end of the T-wave may result in diaphragm movement during the quietest window free of cardiac sounds, minimize acoustic signals not attributable to movement of the diaphragm, and allow expansion of the lung at a time with little to no cardiac interaction. This timing results in cooperation of the breathing cycle with the cardiac cycle, such as the timing used when treating respiratory sinus arrhythmia. A delay in the delivery of PNS after the T-wave (e.g., 100-500 ms) may reduce any residual noise caused by the last heart sound of the cardiac cycle. After determining timing of PNS, method 600 may flow to operation 502 of the method 500 described in FIG. 5. Method 600 may also be performed in identifying or distinguishing PNS contributions in the acoustic signal from the cardiac contributions.

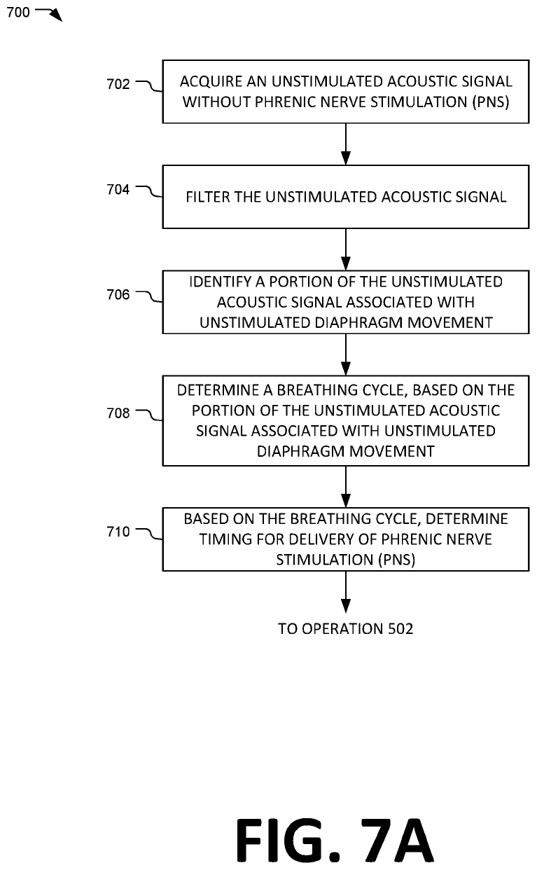

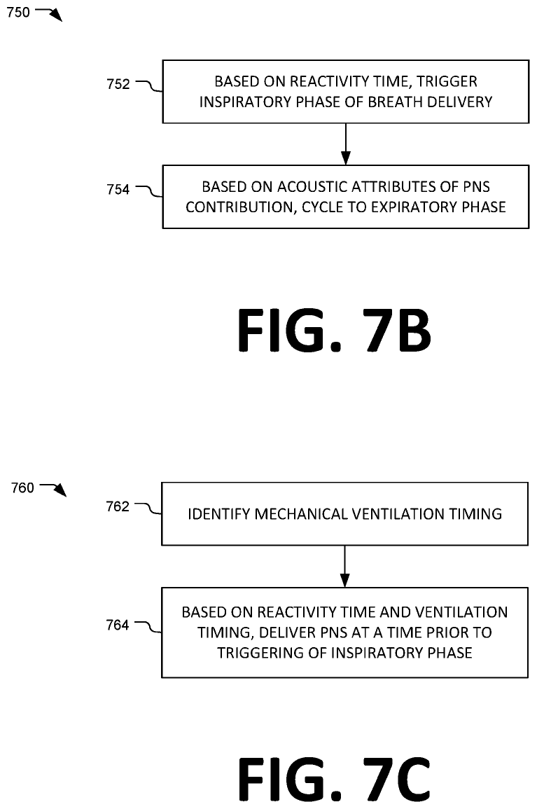

FIG. 7A shows another method 700 for timing phrenic nerve stimulation, based on a natural breathing cycle. At operation 702, an unstimulated acoustic signal is acquired without phrenic nerve stimulation (PNS) (e.g., in the absence of PNS). Without PNS, artifacts in an acoustic signal associated with movement of the diaphragm may be attributable to natural breathing of the patient (e.g., spontaneous breathing or patient effort). At operation 704, the unstimulated acoustic signal is filtered. Filtering of the signal may reduce noise. An ultra-low pass filtering may promote detection of a portion of an acoustic signal associated with natural diaphragm contraction.

At operation 706, a portion of the unstimulated, filtered acoustic signal is identified as associated with movement of the diaphragm. The portion of the acoustic signal associated with diaphragm movement may be identified by filtering out a cardiac cycle. A cardiac cycle may be filtered based on related timing of an ECG and/or EGM, pattern recognition, and/or frequency of repetitive heart sounds over time.

At operation 708, a breathing cycle is determined. The breathing cycle may be determined based on the identified portion of the acoustic signal associated with movement of the diaphragm. For example, sounds caused by movement of the diaphragm may be associated with a contraction of the diaphragm, which is associated with a beginning of an inhalation phase of the breathing cycle. In some examples, relaxation of the diaphragm during exhalation may not be detectable by an acoustic sensor.

At operation 710, timing for delivery of PNS is determined, based on the breathing cycle. If a consistent and/or constant respiratory rate is identified in the patient's natural breathing efforts, PNS may be synchronized with a patient's natural breathing efforts (e.g., during a natural inhalation phase, which corresponds with the portion of the acoustic signal associated with movement of the diaphragm). To correspond with a patient's natural breathing efforts, PNS may be delivered in advance of the natural breathing effort, which varies from patient to patient depending on reactivity time of the diaphragm from PNS (e.g., 100-400 ms). Alternatively, if a natural breathing effort is inconsistent, delivery of PNS may be delayed for a predetermined time lapse to allow the patient time to breath naturally, without PNS. In this example, if a patient does not make a natural effort within the predetermined time lapse, PNS may thereafter be delivered.

Additionally or alternatively, the timing of PNS may be synchronized with breath delivery from the ventilator. For example, the PNS delivery may be timed such that contraction of the diaphragm occurs with a breath delivery (e.g., an inspiratory phase) by the ventilator. Similarly, the exhalation phase of the breath may be synchronized with the end of the contraction of the diaphragm. As an example, if the reactivity time for the patient indicates that contraction of the diaphragm begins 100 ms after the PNS delivery, the PNS system and/or the ventilator may be synchronized such that the inspiratory phase is triggered 100 ms after the PNS delivery. Accordingly, triggering of an inspiratory phase and cycling to an exhalation phase by the ventilator may be based on the timing of the PNS determined through the acoustic analysis. After determining timing of PNS, method 700 may flow to operation 502 of the method 500 described in FIG. 5.

FIG. 7B-7C show methods 750, 760 for synchronizing phrenic nerve stimulation with mechanical ventilation. In FIG. 7B, the method 750 is performed by a ventilator to synchronize breath delivery with PNS delivery. At operation 752, based on the reactivity time determined in method 700, an inspiratory phase of a breath is triggered. For instance, the inspiratory phase of a breath is triggered at a time that is about the reactivity time after the PNS delivery. In some examples, cycling to the expiratory phase may also be based on the acoustic attributes of the PNS contribution to the acoustic signal at operation 754. For instance, a duration of the PNS contribution that resulted from diaphragm contraction may be determined, and cycling of the expiratory phase may be timed to occur at or after that duration.

In FIG. 7C, the method 760 is performed by a PNS controller (which may be part of the ventilator) to synchronize the PNS delivery with the breath delivery from the ventilator. At operation 762, mechanical ventilation timing is identified. The mechanical ventilation timing may be received directly from the ventilator and/or may be determined from the acoustic signal. For instance, the expansion of the lungs due to the mechanical ventilation may be identified in the acoustic signal. For mandatory ventilation modes, a respiratory rate may be set and the inspiratory phase of the breath is triggered at set intervals and such ventilation timing may be determined based on the intervals. At operation 764, PNS is delivered at a time prior to triggering of the inspiratory phase. The triggering time can be determined from the ventilation timing. The PNS is then delivered at a time prior to the triggering based on the reactivity time determined in method 700. For instance, the PNS is delivered at the reactivity time prior to the triggering of the inspiratory phase.

FIG. 8 shows a method 800 for identifying diaphragm movement attributable to phrenic nerve stimulation. At operation 802, a baseline acoustic signal is detected, without phrenic nerve stimulation (PNS). The baseline acoustic signal includes sounds and vibrations detected from natural movement of the internal organs of the body, such as natural diaphragm sounds (e.g., movement of the diaphragm resulting from patient effort, otherwise referred to as natural breathing or spontaneous breathing) and cardiac sounds. If a patient is not breathing on their own, the baseline acoustic signal may not include any portion attributable to diaphragm movement. Cardiac sounds will be present in the baseline acoustic signal so long as the heart is beating. Detection of the baseline acoustic signal without PNS may be performed when PNS is temporarily paused (e.g., PNS was previously delivered to the patient, such as that described at operation 806) or prior to delivery of PNS to the patient.

At operation 804, the baseline acoustic signal is filtered. Filtering of the acoustic signal may remove noise from the acoustic signal. Filtering of the acoustic signal may be similar to that described at operation 506 of FIG. 5.

At operation 806, PNS is delivered. PNS may be delivered according to stimulation parameters and timing otherwise described herein. At operation 808, a stimulated acoustic signal is detected in the presence of PNS. Detection may be performed using an acoustic sensor described herein to detect sounds or vibrations.

At operation 810, the stimulated acoustic signal is filtered. Filtering of the acoustic signal may remove noise from the acoustic signal. Filtering of the acoustic signal may be similar to that described at operation 804 or at operation 506 of FIG. 5. In an example where similar conditions or a similar environment is expected for both the baseline acoustic signal and the stimulated acoustic signal, filtering at operation 804 and operation 810 may be omitted because the baseline acoustic signal and stimulated acoustic signal may include the same or similar background noise. A comparison of the two signals, such as that described below at operation 812, may remove or reduce the same or similar noise between the two signals. If the baseline acoustic signal is filtered at operation 804, then the stimulation acoustic signal should also be filtered at operation 810. Noise may include physiological or non-physiological noise, such as heart sounds from the heartbeat, other heart sounds associated with valve diseases, sounds from other organs, lung movement (e.g., which could result from positive pressure ventilation or diaphragm movement) environmental noise from outside of the patient, speech, etc.

At operation 812, the baseline acoustic signal is compared with the stimulated acoustic signal. If the signals are filtered, then the filtered baseline acoustic signal is compared with the filtered stimulated acoustic signal. The signals may be aligned for the comparison. For example, signals may be aligned based on one or more identified heart sounds, EGM/ECG markers, natural diaphragm movement, or other patterned and/or repeating signals or artifacts included in both the baseline acoustic signal and stimulated acoustic signal. The comparison may be a subtraction of the baseline acoustic signal from the stimulated acoustic signal. A subtraction may result in an acoustic signal associated with artificial movement of the diaphragm (e.g., movement of the diaphragm resulting from PNS) because natural movement of the diaphragm would otherwise be included in both the baseline acoustic signal and the stimulated acoustic signal. Natural movement of the diaphragm may otherwise be identified and/or removed from the comparison based on a secondary monitoring device that monitors the patient's effort.

The comparison may be used to identify a PNS portion of the stimulated acoustic signal associated with artificial movement of the diaphragm. Identification of the PNS portion is described at determination 508 in FIG. 5. The comparison may also allow for identification of respiratory conditions of the patient. For example, frequency (e.g., timing) and shape of peaks of the acoustic signals may be used to determine if a patient is making a spontaneous effort and/or if other respiratory therapies are being delivered. For example, if the acoustics associated with diaphragm movement are more frequent than PNS delivery or two peaks seen in association with diaphragm movement, then the patient may be making a spontaneous effort. Alternatively, a shape of the peak may be different for spontaneous breathing. Other artifacts associated with lung movement may be including in an acoustic signal resulting from a patient that is ventilated (e.g., receiving positive pressure respiratory therapy).

FIG. 9 shows a method 900 for determining or evaluating diaphragm atrophy. At operation 902, phrenic nerve stimulation (PNS) is delivered. At operation 904, an acoustic signal is acquired or detected. Operations 902-904 are similar to other operations described herein for delivery of PNS and detection of an acoustic signal.

At operation 906, a first portion of the acoustic signal associated with diaphragm movement is identified. Identification of the first portion associated with diaphragm movement may be based on pattern recognition, machine learning, markers, and/or a comparison of signals (e.g., method 800 of FIG. 8). For example, frequencies, amplitudes, and/or shapes of the acoustic signal may be used to identify portions of the acoustic signal associated with diaphragm movement. For instance, amplitudes recurring every 3-5 seconds, peaks with lower amplitudes, ramped-up shapes, and/or peaks with a delay from delivery of PNS may be associated with diaphragm movement, rather than noise or heart sounds. Other identification of acoustics associated with diaphragm movement is described herein.

At operation 908, a ratio is determined based on at least the first portion of the acoustic signal. This ratio may be the acoustic-to-electrical PNS ratio discussed above. The ratio may relate an acoustic attribute of the first portion of the acoustic signal with at least one stimulation parameter of the stimulation parameters for PNS at operation 902. The ratio may be an attribute of the acoustic signal that is associated with a certain stimulation parameter. For example, the ratio may relate amplitude of the acoustic signal with voltage of the PNS, duration of a peak of the acoustic signal with pulse width of the PNS, slope of a peak of the acoustic signal with ramp up of a pulse of the PNS, etc. The ratio may change over time. A change in ratio may be due to a physiological change or due to a change in instrumentation. For example, the ratio may change if the diaphragm is atrophying. As another example, the ratio may change over time if a device's integrity changes (e.g., a device, such as the stimulation device, acoustic sensor, battery, processor, etc. malfunctions or is damaged).

At operation 910, an atrophy threshold is determined, based on a moving average of the ratio. A ratio falling below the atrophy threshold may be associated with diaphragm atrophy, other physiological change, or instrumentation dysfunction or repositioning. A moving average of the ratio may be calculated over a set quantity of breaths, heartbeats, and/or time preceding the current ratio. The moving average of the ratio may be calculated over a sufficiently long time window such that occurrences of overlap between the cardiac acoustic sounds and the sounds associated with PNS minimally affect determination of the threshold. The atrophy threshold may be an average over the moving window or an adjustment of the average over the moving window. For example, a percentage of the average may be set as the atrophy threshold (e.g., 50%, 60%, 70%, 75%, 80%, etc.). Accordingly, if the ratio drops below the atrophy threshold, atrophy of the diaphragm may be occurring.

At determination 912, a determination is made as to if the ratio is below the atrophy threshold. If the ratio is determined to be below the atrophy threshold, flow proceeds “YES” to operation 914. At operation 914, a notification is generated. The notification may indicate possible physiological changes of the patient that may cause a decrease in ratio (e.g., diaphragm atrophy, change in compliance, etc.), possible changes in one or more devices (e.g., failure or movement of an acoustic sensor, introduction of background noise, etc.), other potential therapy methods available for the patient, and/or any other information associated with a change in the ratio.

If, alternatively, the ratio is determined to be at or above the atrophy threshold, flow proceeds “NO” back to operation 902. Operations 902-914 may repeat as required or desired. For example, if no diaphragm atrophy is determined, based on the ratio, then PNS may continue to be delivered without generating a notification and the diaphragm may continue to be evaluated. If diaphragm atrophy is determined, based on the ratio, then a notification may be generated and PNS may continue to be delivered.

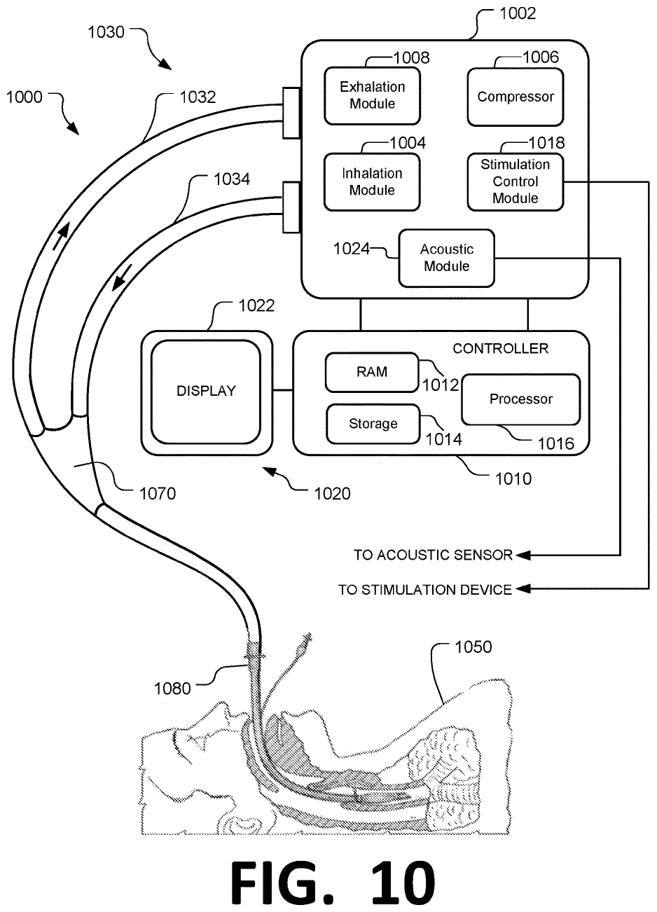

FIG. 10 is a diagram illustrating an example of a ventilator 1000 connected to a patient 1050 and a stimulation device. Ventilator 1000 includes a pneumatic system 1002 (also referred to as a pressure generating system 1002) for circulating breathing gases to and from patient 1050 via the ventilation tubing system 1030, which couples the patient to the pneumatic system via an invasive (e.g., endotracheal tube, as shown) or a non-invasive (e.g., nasal mask) patient interface.

Ventilation tubing system 1030 may be a two-limb (shown) or a one-limb circuit for carrying gases to and from the patient 1050. In a two-limb example, a fitting, typically referred to as a wye-fitting or wye 1070, may be provided to couple a patient interface 1080 to an inhalation limb 1034 and an exhalation limb 1032 of the ventilation tubing system 1030.

Pneumatic system 1002 may have a variety of configurations. In the present example, pneumatic system 1002 includes an exhalation module 1008 coupled with the exhalation limb 1032 and an inhalation module 1004 coupled with the inhalation limb 1034. Compressors or other source(s) of pressurized gases 1006 (e.g., air, oxygen, and/or helium) are coupled with inhalation module 1004 to provide a gas source for ventilatory support via inhalation limb 1034. Stimulation control module 1018 may provide voltage to a stimulation device (e.g., to independently control electrodes on the stimulation device), and acoustic module 1024 may analyze acoustic signals, as described herein. The pneumatic system 1002 may include a variety of other components, including mixing modules, valves, sensors, tubing, accumulators, filters, etc.

Controller 1010 is operatively coupled with pneumatic system 1002, signal measurement and acquisition systems, and an operator interface 1020 that may enable an operator to interact with the ventilator 1000 (e.g., change ventilator settings, select operational modes, view monitored parameters, etc.). Controller 1010 may include memory 1012, one or more processors 1016, storage 1014, and/or other components of the type found in command and control computing devices. In the depicted example, operator interface 1020 includes a display 1022 that may be touch-sensitive and/or voice-activated, enabling the display 1022 to serve both as an input and output device.

The memory 1012 includes non-transitory, computer-readable storage media that stores software that is executed by the processor 1016 and which controls the operation of the ventilator 1000. In an example, the memory 1012 includes one or more solid-state storage devices such as flash memory chips. The processor 1016 may be configured to control attributes of the electrodes on a stimulation device. In an alternative example, the memory 1012 may be mass storage connected to the processor 1016 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 1016. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra-or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

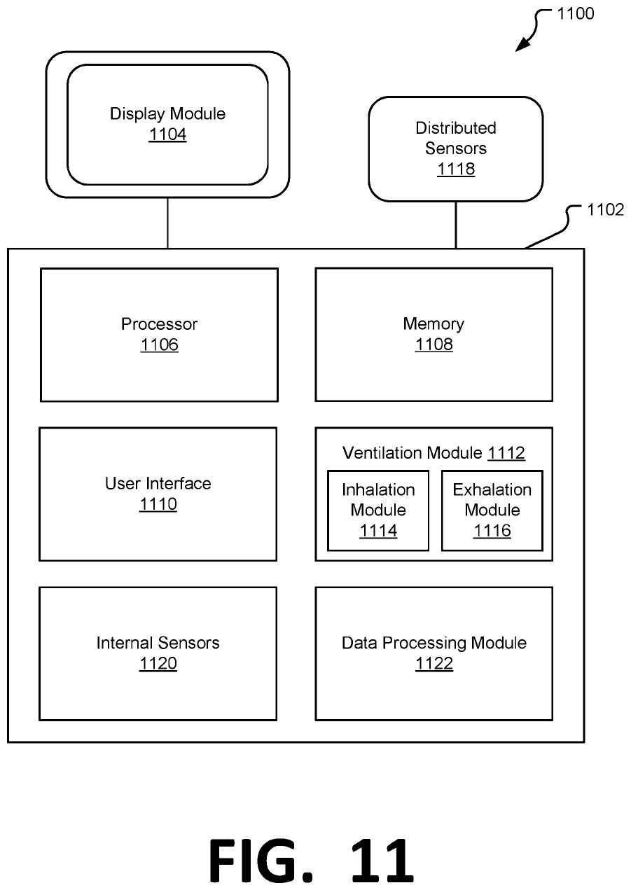

FIG. 11 is a block-diagram illustrating an example of a ventilatory system 1100. Ventilatory system 1100 includes ventilator 1102 with its various modules and components. That is, ventilator 1102 may further include, among other things, memory 1108, one or more processors 1106, user interface 1110, and ventilation module 1112 (which may further include an inhalation module 1114 and an exhalation module 1116). Memory 1108 is defined as described above for memory 1012. Similarly, the one or more processors 1106 are defined as described above for one or more processors 1016. Processors 1106 may further be configured with a clock whereby elapsed time may be monitored by the ventilatory system 1100. The ventilator 1102 may be connected to distributed sensors 1118, such as the sensors discussed herein.

The ventilatory system 1100 may also include a display module 1104 communicatively coupled to ventilator 1102. Display module 1120 (otherwise referred to as operator interface 1110) provides various input screens, for receiving input, and various display screens, for presenting useful information. Inputs may be received from a clinician. The display module 1120 is configured to communicate with user interface 1110 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 1102 may be provided, for instance by a wheel, keyboard, mouse, or other suitable interactive device. Thus, user interface 1110 may accept commands and input through display module 1104. Display module 1104 may also provide useful information in the form of various ventilatory data regarding the physical condition of a patient and/or a prescribed respiratory treatment. The useful information may be derived by the ventilator 1102, based on data collected by a data processing module 1122, and the useful information may be displayed in the form of graphs, wave representations (e.g., a waveform), pie graphs, numbers, or other suitable forms of graphic display. For example, the data processing module 1122 may be operative to determine ventilation settings (otherwise referred to as ventilatory settings, or ventilator settings) associated with a stimulation device for nerve stimulation, etc., as detailed herein.

Ventilation module 1112 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with stimulating a phrenic nerve with a stimulation device. In some cases, certain ventilation settings may be adjusted based on the exhalation flow, e.g., to optimize the prescribed treatment.

Ventilation module 1112 may further include an inhalation module 1114 configured to deliver gases to the patient and an exhalation module 1116 configured to receive exhalation gases from the patient, according to ventilation settings that may be based on the exhalation flow. As described herein, inhalation module 1114 may correspond to the inhalation module 1004, 1114, or may be otherwise coupled to source(s) of pressurized gases (e.g., air, oxygen, and/or helium), and may deliver gases to the patient. As further described herein, exhalation module 1116 may correspond to the exhalation module 1008 and 1116, or may be otherwise coupled to gases existing the breathing circuit.

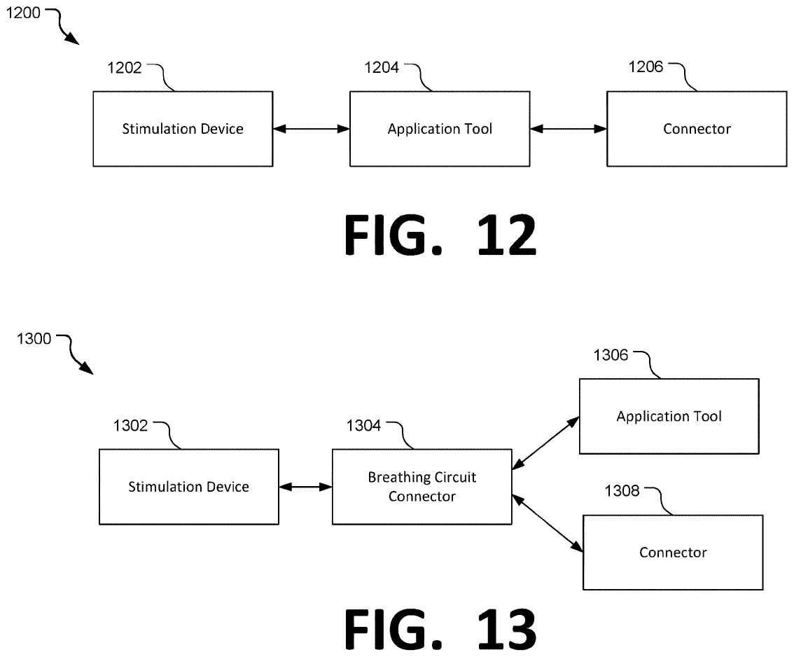

FIG. 12 shows a phrenic nerve stimulation system 1200, including a stimulation device 1202, an application tool 1204, and a connector 1206. The stimulation device 1202 is a device capable of stimulating one or more phrenic nerves, such as a phrenic nerve lead, which may be inserted into a body of a patient intravenously.

The application tool 1204 is assists with inserting, implanting, removing, or otherwise moving the stimulation device 1202 within the body of the patient while the application tool 1204 remains outside of the body of the patient. The application tool 1204 enables a stylet to be inserted into, removed from, or otherwise moved inside of the stimulation device 1202 to cause the stimulation device 1202 to change shape and/or be secured inside of the body of the patient.

The connector 1206 may be connects independent leads for one or more of the electrodes on the stimulation device 1202. Each lead of the connector 1206 may independently energize an electrode at the stimulation device 1202 to which the lead is electrically coupled. For example, if the stimulation device 1202 has eight electrodes, the connector 1206 may have eight independent leads electrically coupled to each of the eight electrodes. The connector 1206 may be electrically couplable to a controller to independently energize each electrode on the stimulation device 1202 at each lead. The controller may be a component of a ventilator (e.g., stimulation lead control module 1018 of ventilator 1002). Alternatively, the controller may be a stand-alone nerve stimulation controller, may be incorporated into another other device such as a patient monitor, or may be shared with another device such as an acoustic sensor and/or an external stimulation pad. The controller may include a processor and memory with instructions that, when executed by the processor, cause the operations discussed herein to be performed.

FIG. 13 shows another example stimulation system 1300 including a stimulation device 1302, an application tool 1306, and a connector 1308. Unlike the stimulation system 1200 of FIG. 12, the stimulation system 1300 of FIG. 13 may be coupled to a breathing circuit connector 1304 integrated into the breathing circuit, which is then couplable to the application tool 1306 and the connector 1308 via the breathing circuit connector 1304.

The stimulation device 1302 may be any device capable of stimulating one or more phrenic nerves, such as at electrodes coupled to an inflatable balloon, which may be insertable into an esophagus of a patient. The application tool 1306 may be similar to the application tool 1204 described in FIG. 12. Additionally or alternatively, the application tool 1306 may enable inflation and deflation of an inflatable balloon of a stimulation device 1302 (e.g., to secure the stimulation device 1302 inside the body of the patient). The application tool 1306 may be configured to interface with the stimulation device 1302 at the breathing circuit connector 1304 (e.g., at a port of the breathing circuit connector 1304). The connector 1308 may be similar to the application tool 1306 described in FIG. 13. Additionally or alternatively, the connector 1308 may be configured to interface with the electrodes of the stimulation device 1302 at the breathing circuit connector 1304 (e.g., at a port of the breathing circuit connector 1304).

Although the present disclosure discusses the implementation of these techniques in the context of phrenic nerve stimulation, stimulation of any nerve or nerves in the body of a patient is appreciated. Additionally, although the stimulation devices are described with specific uses and functions for nerve stimulation, any device with one or more leads may be used. Any sound, vibration, or movement detectable by an acoustic sensor may be analyzed via the methods and systems described herein.

Placement of the stimulation device in any orifice in the body is appreciated, beyond the placement described with respect to veins and an esophagus of a patient. For example, the stimulation device can be adapted for any vein or tube in the body to excite any nerve nearby.

Further, the techniques introduced above may be implemented for a variety of medical devices or devices utilizing nerve stimulation. A person of skill in the art will understand that the technology described in the context of a stimulation device and/or acoustic sensor for human patients could be adapted for use with other systems such as ventilators for non-human patients or general gas transport systems.

Those skilled in the art will recognize that the methods and systems of the present disclosure may be implemented in many manners and as such are not to be limited by the foregoing aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A computer-implemented method for providing phrenic nerve stimulation, the method comprising:
delivering phrenic nerve stimulation, at a first time based on stimulation parameters, to a phrenic nerve to cause movement of a diaphragm;
acquiring, from an internal acoustic sensor, an acoustic signal including contributions from diaphragm movement and cardiac movement;
identifying a first portion of the acoustic signal associated with the diaphragm movement and a second portion of the acoustic signal associated with cardiac movement;
based the first portion of the acoustic signal, adjusting the stimulation parameters; and
delivering phrenic nerve stimulation, at a second time, based on the adjusted stimulation parameters.

2. The method of claim 1, further comprising determining, based at least on the first portion of the acoustic signal, that the movement of the diaphragm caused by the phrenic nerve stimulation is associated with one of a hiccup or a low tidal volume.

3. The method of claim 2, wherein adjusting the stimulation parameters is based on determining that the movement of the diaphragm is associated with one of the hiccup or the low tidal volume.

4. The method of claim 1, wherein the acoustic sensor is part of at least one of an implantable cardioverter-defibrillator (ICD), pacemaker, or neurostimulator.

5. The method of claim 1, further comprising:
determining a ratio based on at least one acoustic attribute the first portion of the acoustic signal and at least one stimulation parameter of the stimulation parameters;
calculating an atrophy threshold, based on a moving average of the ratio;
determining that the ratio is below the atrophy threshold; and
generating a notification for diaphragm atrophy.

6. The method of claim 5, wherein the acoustic attribute is a peak amplitude of the first portion of the acoustic signal.

7. The method of claim 1, the method further comprising triggering an inspiratory phase of a breath based on one or more acoustic attributes of the first portion of the acoustic signal.

8. A computer-implemented method for providing phrenic nerve stimulation, the method comprising:
acquiring, by an internal acoustic sensor, a baseline acoustic signal;
delivering phrenic nerve stimulation to a phrenic nerve, based on stimulation parameters, to cause artificial movement of a diaphragm;
subsequent to delivering the phrenic nerve stimulation, acquiring, by the internal acoustic sensor, a stimulated acoustic signal;
comparing, by a phrenic nerve stimulation controller, the stimulated acoustic signal and the baseline acoustic signal; and
identifying a portion of the stimulated acoustic signal attributable to the artificial movement of the diaphragm.

9. The method of claim 8, wherein the acquiring the baseline acoustic signal occurs during a period where no phrenic nerve stimulation is delivered.

10. The method of claim 8, the method further comprising adjusting at least one of the stimulation parameters based on the portion of the stimulated acoustic signal attributable to the artificial movement of the diaphragm.

11. The method of claim 10, wherein the stimulation parameters include at least one of:
a frequency;
a timing;
an amplitude; or
a pulse width.

12. The method of claim 11, wherein the phrenic nerve stimulation controller is integrated into a medical ventilator.

13. A phrenic nerve stimulation system comprising:
an acoustic sensor internal to a body;
a phrenic nerve stimulation device;
a processor;
memory storing instructions that, when executed by the processor, cause the system to perform a set of operations comprising:
delivering a first phrenic nerve stimulation, via the stimulation device, to at least one phrenic nerve of the body according to stimulation parameters;
acquiring, via the acoustic sensor, an acoustic signal of sounds from within the body;
identifying a portion of the acoustic signal associated with diaphragm movement;
based on the identified portion of the acoustic signal, adjusting the stimulation parameters; and
delivering a second phrenic nerve stimulation, via the stimulation device, based on the adjusted stimulation parameters.

14. The phrenic nerve stimulation system of claim 13, further comprising an implantable cardioverter-defibrillator (ICD), wherein the acoustic sensor is part of the ICD.

15. The phrenic nerve stimulation system of claim 13, further comprising a ventilator, and wherein the operations further comprise:
based on the identified portion of the acoustic signal, determining a reactivity time between the delivery of the first phrenic nerve stimulation and the diaphragm movement; and
causing an inspiratory breath to be triggered at about the reactivity time after delivery of the second phrenic nerve stimulation.
